(19) United States
(12) Patent Application Publication
(10) Pub. No.: US 2021/0350686 A1
(43) Pub. Date: Nov. 11, 2021

(54) PROXIMITY TRACKING SYSTEM FOR MONITORING SOCIAL DISTANCING

(71) Applicant: EMBEDTEK, LLC, Waukesha, WI (US)

(72) Inventors: Kent Tabor, Pewaukee, WI (US); Ryan Rethwisch, Elm Grove, WI (US)

(21) Appl. No.: 17/316,626

(22) Filed: May 10, 2021

Related U.S. Application Data
(60) Provisional application No. 63/021,840, filed on May 8, 2020.
(60) Provisional application No. 63/042,699, filed on Jun. 23, 2020.

(51) Int. Cl. G08B 21/02; G10L 25/51; G08B 21/18; G01S 11/02; G01S 1/02

(52) U.S. Cl. CPC G08B 21/02; G10L 25/51; G01S 1/02; G01S 11/02; G08B 21/182

(57) ABSTRACT

A proximity monitor includes an electronic processor, a proximity detector, and a memory. The proximity detector is to receive a proximity signal, and the electronic processor is to determine a time-of flight measurement for the proximity signal, determine a proximity measure based on the time-of-flight, identify a proximity event based on the proximity measure, and store a leg entry for the proximity event in the memory, the log entry identifying a first identifier associated with the proximity monitor and a second identifier associated with a sender of the proximity signal.

### RELATED APPLICATIONS

This patent application claims priority to U.S. Provisional Patent Application No. 63/021,840, filed May 8, 2020, and U.S. Provisional Patent Application No. 63/042,699 filed Jun. 23, 2020, the entire contents of each of which are hereby incorporated by reference.

### FIELD

Embodiments of the invention relate to monitoring populations for potential pathogen transmission and, more particularly, to a proximity tracking system useful for monitoring social distancing and that allows for evaluation of potential pathogen transmission (“contact tracing”).

### BACKGROUND

Certain pathogens are transmitted between individuals that come in close contact with one another. For example, if an infected individual coughs or sneezes droplets containing the pathogen may be transferred to other nearby individuals. Example pathogens may include bacteria, fungi, or viruses. One such coronavirus responsible for a worldwide pandemic starting in 2019 results in a disease named COVID-19. One technique for reducing the spread of pathogens transferred by droplets is to maintain an increased distance between individuals, a practice known as social distancing. One problem associated with controlling the spread of pathogens is that an infected individual may be asymptomatic. Social distancing is a barrier to pathogen transmission, but it is not completely effective, because it is dependent on human behavior.

Pandemics can have drastic economic consequences as businesses, schools, government operations may be shut down to prevent the spread of the pathogen. In some instances, an entire facility maybe shut down, even if only a small number of infected individuals are identified. In some facilities, maintaining adequate social distancing is difficult. Tracking contacts between individuals may be effective in identifying individuals that have been in contact with another individual found to be infected to facilitate quarantining potentially infected individuals without shutting down the facility. However, such monitoring techniques may use expensive equipment, such as video surveillance, smartphones, or the like, making them cost prohibitive for some applications. In addition, contact tracking may also raise privacy concerns for the individuals being monitored.

### SUMMARY

Proximity tracking is facilitated through proximity monitors that employ time-of flight to accurately measure proximity between individuals. Techniques for implementing proximity measurement may include polling techniques or signal beacon techniques. Audio frequency and/or radio frequency signals may be used for time-of flight measurement. Proximity warnings can serve as a reminder to increase distancing, reducing the duration of a distance breach. Proximity events and duration may be logged between individuals to facilitate intervention should an individual be diagnosed with a communicable disease. In some embodiments, additional sensors are integrated into the proximity monitor, such as temperature sensors or cough sensors to detect symptomology. Proximity monitors may be provided on wearable items, such as personal protective equipment or clothing items, an ID badge, wristband, lanyard, or the like.

In one embodiment, a proximity monitor includes an electronic processor, a proximity detector, and a memory. The proximity detector is to receive a proximity signal, and the electronic processor is to determine a time-of flight measurement for the proximity signal, determine a proximity measure based on the time-of-flight, identify a proximity event based on the proximity measure, and store a leg entry for the proximity event in the memory, the log entry identifying a first identifier associated with the proximity monitor and a second identifier associated with a sender of the proximity signal.

In another embodiment, a method for monitoring proximity includes receiving, by a proximity detector a proximity signal, determining, by an electronic processor, a time-of flight measurement for the proximity signal, determining, by the electronic processor, a proximity measure based on the time-of-flight, identifying, by the electronic processor, a proximity event based on the proximity measure, and storing, by the electronic processor, a log entry for the proximity event in a memory, the log entry identifying a first identifier associated with the proximity monitor and a second identifier associated with a sender of the proximity signal.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is a diagram illustrating a proximity tracking system, according to some embodiments.

FIG. 2 is a block diagram of a proximity monitor, according to some embodiments.

FIGS. 3 and 4 are flowcharts of methods performed by a computing device for proximity tracking, according to some embodiments.

FIG. 5 is a diagram illustrating headgear incorporating the proximity monitor 110, according to some embodiments.

FIG. 6 is a diagram illustrating an identification (ID) badge incorporating the proximity monitor, according to some embodiments.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Before any embodiments of the invention are explained in detail, it is to be understood that the invention is not limited in its application to the details of construction and the arrangement of components set forth in the following description or illustrated in the accompanying drawings. The invention is capable of other embodiments and of being practiced or of being carried out in various ways.

One or more embodiments are described and illustrated in the following description and accompanying drawings. These embodiments are not limited to the specific details provided herein and may be modified in various ways. Furthermore, other embodiments may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way, but may also be configured in ways that are not listed. Furthermore, some embodiments described herein may include one or more electronic processors configured to perform the described functionality by executing instructions stored in non-transitory, computer-readable medium. Similarly, embodiments described herein may be implemented as non-transitory, computer-readable medium storing instructions executable by one or more electronic processors to perform the described functionality. As used herein, “non-transitory computer-readable medium” includes all computer-readable media but does not consist of a transitory, propagating signal. Accordingly, non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

Also, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms such as first and second, top and bottom, and the like may be used herein solely to distinguish one entity or action from another entity or action without necessarily requiring or implying any actual such relationship or order between such entities or actions.

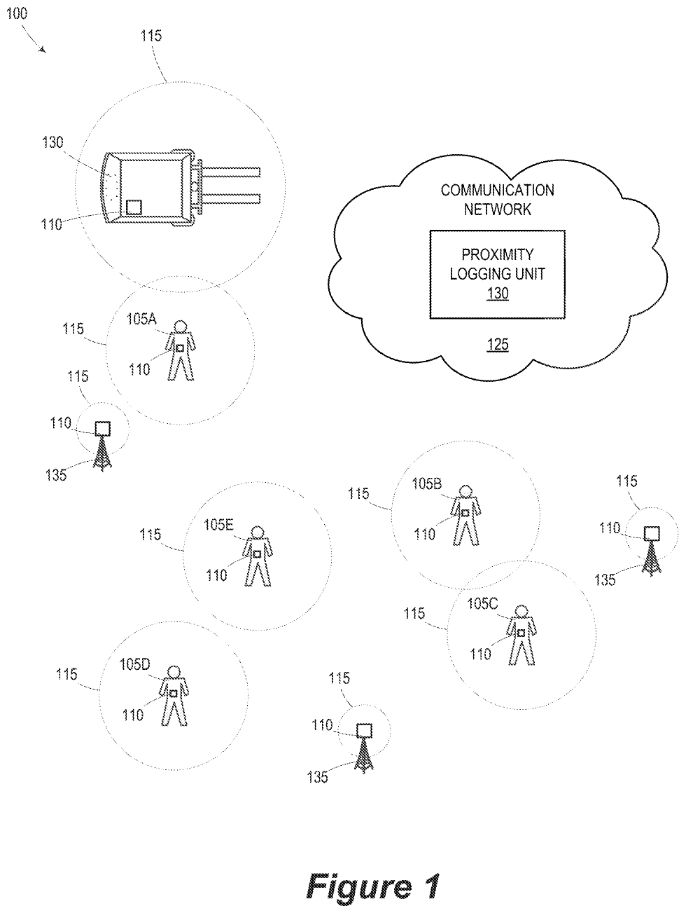

FIG. 1 is a diagram illustrating a proximity tracking system 100, according to some embodiments. The proximity tracking system 100 is used to identify proximity events between individuals 105A-105E. In some embodiments, the individuals 105A-105E are associated with a particular facility, work group, etc. Each individual 105A-105E is equipped with a proximity monitor 110 that determines if another individual comes within a proximity threshold 115. In some embodiments, the proximity threshold 115 is determined based on a social distancing threshold related to a pathogen transmission distance. For example, if a known pathogen, such as COVID-19, is known to have a 6-foot transmission radius, the proximity threshold 115 may be set at or above the 6-foot range. The proximity monitors 110 detect and log proximity events and communicate the proximity events to a proximity logging unit 120 through interfaces with a communication network 125 (e.g., WiFi, BLUETOOTH®, cellular, Internet, RF, Ultra Wideband (UWB), or the like). In some embodiments, the proximity monitors 110 are configured to communicate their local proximity event logs to the proximity logging unit 120 using a wireless protocol over the communication network 125. In some embodiments, the proximity monitors 110 are connected using a wired connection to the proximity logging unit 120 periodically to upload their proximity event logs. In some embodiments, the proximity logging unit 120 may be implemented as a cloud-based resource using a virtual computing environment. In some embodiments, the proximity logging unit 120 is a server.

In some embodiments, a proximity monitor 110 is associated with a vehicle 130, such as a fork truck, or other vehicle. In some embodiments, the proximity monitor 110 is mounted to the vehicle 130. In some embodiments, the proximity monitor 110 is carried by a driver of the vehicle 130. A proximity threshold 115 is associated with the proximity monitor 110 on the vehicle 130. In some embodiments, the proximity threshold 115 for the vehicle 130 is different than the proximity threshold 115 for individuals 105A-105E. In some embodiments, the proximity monitor 110 on the vehicle 130 is employed to implement physical safety distancing, while the proximity monitors 110 for individuals 105A-105E are employed for contract tracing.

In some embodiments, a proximity monitor 110 is associated with a stationary reference point 135. Example stationary reference points 135 include entry or egress points from an area or building, a specific area with enhanced distancing protections, such as barriers, or the like. In some embodiments, the proximity threshold 115 for the stationary reference points 135 is different than the proximity threshold 115 for individuals 105A-105E. In some embodiments, the proximity monitors 110 associated with stationary reference points 135 can trigger actions, such as changing proximity thresholds 115, enabling or disabling tracking, etc.

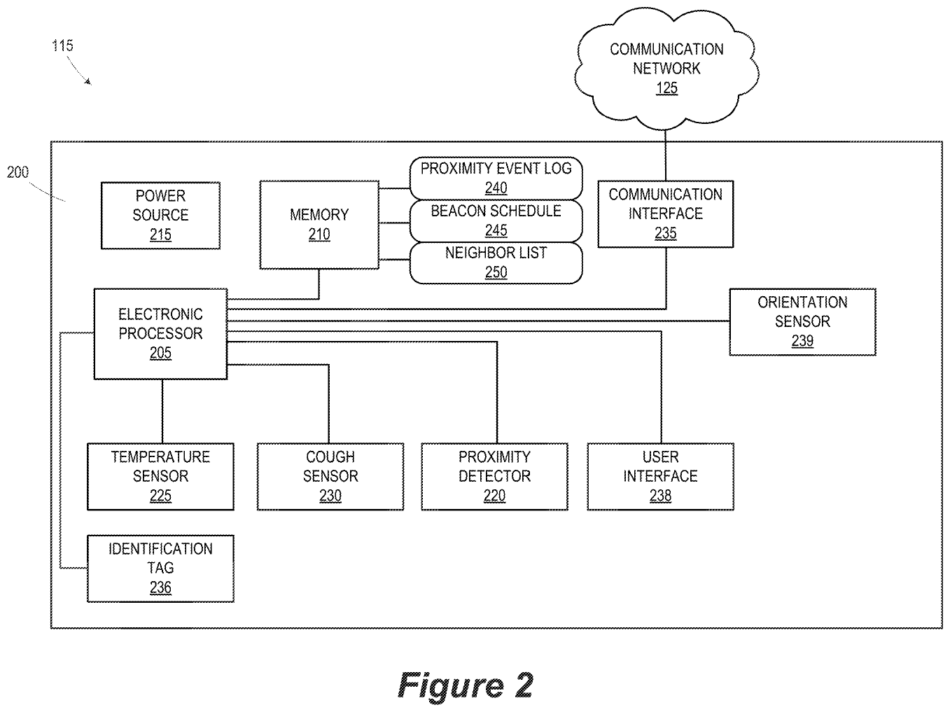

FIG. 2 is a block diagram of a proximity monitor 110, according to some embodiments. Each proximity monitor 110 may contain a housing 200. Such a housing 200 may be manufactured with any suitable materials for the environment in which the individuals 105A-E are present. As illustrated in FIG. 2, the proximity monitor 110 includes an electronic processor 205, a memory 210, a power source 215, a proximity detector 220, a temperature sensor 225, a cough sensor 230, a communication interface 235, an identification tag 236, a user interface 238, and an orientation sensor 239. It should be appreciated that proximity monitor 110 may include any of numerous other types of sensors in addition to or instead of the above-described sensors. Not all sensors may be present in some embodiments. The memory 210 includes read only memory (ROM), random access memory (RAM), other non-transitory computer-readable media, or a combination thereof. The electronic processor 205 is configured to communicate with the memory 210 to store data and retrieve stored data. The electronic processor 205 is configured to receive instructions and data from the memory 210 and execute, among other things, the instructions. In particular, the electronic processor 205 executes instructions stored in the memory 210 to perform the methods described herein. The power source 215 provides power to the various components of the proximity monitor 110. In some embodiments, the memory 210 stores a proximity event log 240 that records events from the proximity detector 220, the temperature sensor 225, or the cough sensor 230. In some embodiments, the power source 215 includes a rechargeable device, such as a battery, a capacitor, a super capacitor, or the like. The power source 215 may charge rechargeable device using inductive charging or energy harvesting. In some embodiments, the power source 215 includes a replaceable battery. In some embodiments, the orientation sensor 239 includes an accelerometer, magnetometer, mercury switch, gyroscope, compass, or some combination thereof. In some embodiments, the orientation sensor 239 is an inertial measurement unit (IMU), such as those used in drones or other navigation applications. In some embodiments, the orientation sensor 239 includes a magnetic compass or a pressure sensor.

The communication interface 235 allows for communication between the electronic processor 205 and an external device, such as the proximity logging unit 120, over the communication network 125. In some embodiments, the communication interface 235 may include separate transmitting and receiving components. In some embodiments, the communication interface 235 is a wireless transceiver that encodes information received from the electronic processor 205, such as the proximity event log 240, into a carrier wireless signal and transmits the encoded wireless signal to the monitoring unit 120 over the communication network 125. The communication interface 235 also decodes information from a wireless signal received from the proximity logging unit 120 over the communication network 125 and provides the decoded information to the electronic processor 205.

In some embodiments, the temperature sensor 225 or the cough sensor 230 generate events independently from the proximity detector 220. If a temperature reading exceeds a temperature threshold, a temperature event may be logged. In some embodiments, the cough sensor 230 includes a microphone or a vibration sensor that compares an input signal to one or more cough signatures to identify if a user is coughing. The cough sensor 230 may identify a frequency of or vibration of detected coughs and log a coughing event if the frequency exceeds a cough threshold. If the proximity detector 110 is not equipped to send an event signal to the proximity logging unit 120 in real time, the proximity monitor 110 may issue an audible, visual, or vibration alert telling the user to seek immediate intervention.

In some embodiments, the user interface 238 includes one or more of input buttons, a display, a visual indicator (e.g., LED light), a vibration indicator, an audible indicator, or the like. In some embodiments, the user interface 238 is employed to set parameters for the proximity monitoring, such as the proximity threshold 115.

In some embodiments, the proximity monitor 110 is implemented in a handheld or wearable device, such as a laptop computer, a handheld computer, a tablet computer, a mobile device, a telephone, a personal data assistant, a music player, a game device, a wearable computing device, and the like. In some embodiments, the proximity monitor 110 is attached to or incorporated into a wearable device, such as an ID badge, a piece of personal protective equipment, safety glasses, a belt, a wristband, a necklace, a clothing item, headgear, a keychain, a lanyard, or the like.

In general, the proximity detector 220 employs a time-of flight detection technique to accurately measure proximity between the individuals 105A-105E or between individuals 105A-105E and vehicles 130. Techniques for implementing proximity measurement may include polling techniques or one-way beacon techniques. Audio frequency and/or radio frequency signals may be used for time-of flight measurement. In some embodiments, the proximity monitor 110 uses RF triangulation to supplement the accuracy of time-of-flight technology. In some embodiments, the proximity monitor 110 uses non synchronized transmissions with error handling collision mitigation algorithms.

In some embodiments, the proximity detector 220 uses audio frequency techniques for measuring time-of flight. In this embodiment, the proximity detector 220 includes an audio transmitter (e.g., speaker) and an audio receiver (e.g., microphone). In some embodiments, the audio frequency range used for proximity detection is not in the range in which humans are able to detect the sounds. For example, a range of 20 kHz to 35 kHz may be used. In some embodiments, an audible tone is used. Each proximity detector 220 is assigned a transmission time slot in a beacon schedule 245 stored in the memory 210 and maintained by the proximity logging unit 120. Each individual 105A-105E is assigned a unique identifier, which is stored in the beacon schedule 245 with the assigned time slot.

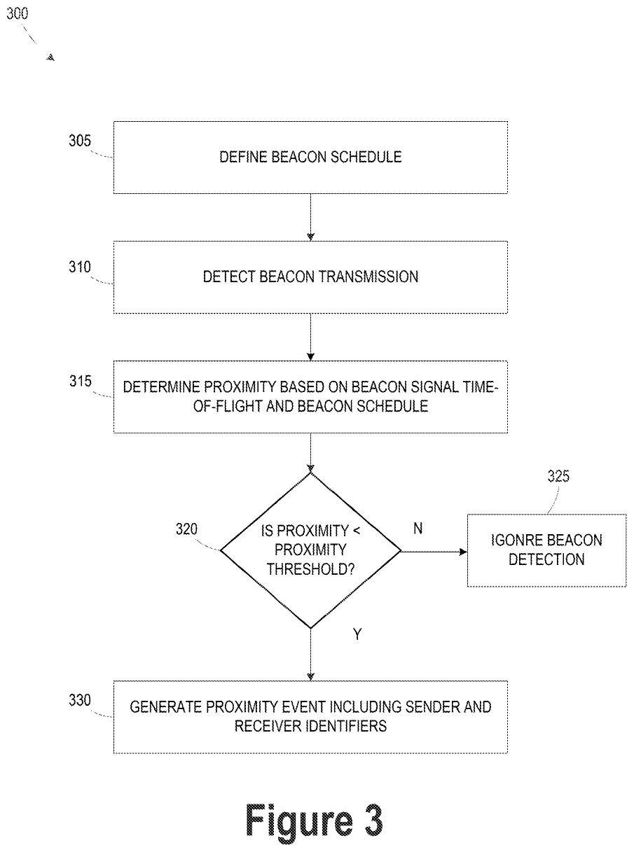

FIG. 3 is a flowchart of a method 300 performed by a computing device for proximity tracking, according to some embodiments. In some embodiments, the method 300 is performed by the electronic processor 205 of the proximity monitor 110. In block 305, a beacon schedule 245 is defined. In some embodiments, the proximity logging unit 120 defines the beacon schedule 245 and transmits it to the proximity monitors 110. In some embodiments, the beacon schedule 245 is programmed into the proximity monitors 110 during a configuration cycle.

In block 310, the proximity detector 220 detects a beacon transmission. For example, the proximity detector 220 may include a microphone or vibration transducer and a signal detector that registers a specific tone. In some embodiments, the signal detector is implemented by the electronic processor 205 based on the received microphone signal.

In block 315, the proximity monitor 110 determines a proximity measure based on a time-of flight for the beacon signal using the beacon schedule 245. Since, the transmission time is defined by the beacon schedule 245, the time-of flight is defined by the time interval between the transmission time and the detection time. The proximity measure is determined by multiplying the time-of flight by the speed of the signal (e.g., the speed of sound). In some embodiments, the speed of sound parameter is compensated based on ambient temperature.

Proximity=(Receive Time-Scheduled Transmission Time)*Signal Speed

In block 320, the proximity monitor 110 determines if the proximity measure is less than the proximity threshold 115. If the proximity measure is not less than the proximity threshold 115, the proximity monitor 110 ignores the beacon detection in block 325. If the proximity measure is less than the proximity threshold 115, the proximity monitor 110 generates a proximity event in block 330. The proximity monitor 110 stores an entry in the proximity event log 240 including an identifier of the sending proximity monitor 110, an identifier of the receiving proximity monitor 110, and a time stamp. In some embodiments, the proximity monitor 110 may also log the proximity measure. In embodiments where the proximity monitor 110 includes a temperature sensor 225 or a cough sensor 235, the temperature or cough frequency of the user associated with the proximity monitor 110 that detects the proximity event is logged with the proximity data. In embodiments where the proximity monitor 110 includes an orientation sensor, the orientation of the user is stored in the proximity event log 240. Information from multiple proximity event logs 240 may be combined to determine whether individuals associated with the same proximity event were facing each other. In general, the beacon schedule 245 is arranged so that no time slots overlap, such that if a particular proximity detector 220 detects a beacon transmission, the identity of the sender is not ambiguous. In some embodiments, different beacon tones may be used to generate overlapping schedules. For example, different subsets of the proximity monitors 110 may be assigned different tones or tones with different data modulated on the tone signal, where the transmission windows for each member of a particular subset do not overlap. A particular proximity detector 220 may detect a first tone corresponding to a proximity monitor 110 in one subset and a second tone from a proximity monitor 110 in a different set during the same time window, but the specific tone frequency or modulated data may be used with the beacon schedule 245 to identify the unique sending proximity monitor 110.

The example of FIG. 3 uses audio signals. Given the speed of RF signals, it may be difficult to achieve adequate clock synchronization to facilitate one-way time-of flight measurement. However, if such clock synchronization is available, RF signals may be used in the method 300 of FIG. 3.

In some embodiments, the proximity detector 220 uses radio frequency techniques for measuring round-trip time-of flight. In this embodiment, the proximity detector 220 includes a radio frequency transceiver. In some embodiments, the transceiver components are shared with the communication interface 235. In some embodiments, the proximity detector 220 includes a transceiver communicating using UWB protocols.

FIG. 4 is a flowchart of a method 400 performed by a computing device for proximity tracking, according to some embodiments. In some embodiments, the method 400 is performed by the electronic processor 205 of the proximity monitor 110. In block 405, the proximity detector 220 identifies neighboring proximity monitors 110. In some embodiments, the proximity detector 220 sends a broadcast poll message, and any proximity units 110 within range send an initial response. The broadcast poll message may be a general message that does not address particular recipients. In some embodiments, each proximity monitor 110 stores a neighbor list 250 (see FIG. 2) that indicates the other proximity monitors 110 it has encountered, such as those responding to the broadcast poll message.

Based on a signal strength parameter associated with the initial responses to the broadcast poll message, the proximity detector 220 can determine a rough range of the nearby proximity units 110. The range provided by signal strength is generally highly variable and not sufficiently accurate for identifying proximity events as described herein.

In block 410, the proximity detector 220 sends a directed poll signal to a particular proximity unit 110 (e.g., specifically addressed message) in the neighbor list 250. In some embodiments, the proximity monitor uses the rough range estimate from the responses to the broadcast poll message to filter the neighbor list 250 to exclude proximity units 110 greater than a certain distance away (e.g., two-three times the proximity threshold 115) from directed polling.

In response to the directed poll signal, the particular proximity unit 110 responds with a directed response signal in block 415 (e.g., addressed response message).

In block 420, the proximity detector 220 determines a proximity measure based on the round-trip time delay between the directed poll signal and the directed response signal and the speed of the RF signal. In general, the latency associated with the nearby proximity unit 110 receiving the directed poll signal and sending the directed response signal can be quantified and is generally fixed. This latency metric is subtracted from the round-trip time delay when determining the proximity measure:

$$Proximity = \frac{Round - trip Time - Latency}{2} * Signal Speed$$

In block 425, the proximity monitor 110 determines if the proximity measure is less than the proximity threshold 115. If the proximity measure is less than the proximity threshold 115, the proximity monitor 110 generates a proximity event in block 430. The proximity monitor 110 stores an entry in the proximity event log 240 including an identifier of the sending proximity monitor 110, an identifier of the receiving proximity monitor 110, and a time stamp. In some embodiments, the proximity monitor 110 also logs the proximity measure. In embodiments, where the proximity monitor 110 includes a temperature sensor 225 or a cough sensor, the temperature or cough frequency of the user associated with the proximity monitor 110 that detects the proximity event is logged with the proximity data. In some embodiments, the proximity monitor 110 transmits proximity measure messages to other proximity monitors 110 using a non-addressed broadcast message regardless of the identification of a proximity event. If a proximity monitor 110 receives a proximity measure message for neighboring proximity monitors 110 not in the neighbor list 250, new entries are added. Using the received proximity measure messages, a proximity measure between a given proximity monitor 110 and other proximity monitors 110 in the neighbor list 250 may be estimated without directed polling using triangulation. A flag in the neighbor list 250 may indicate if the proximity measure for a particular neighbor was generated by direct measurement or estimation (e.g., triangulation).

After generating the proximity event in block 430, the proximity monitor 110 broadcasts a proximity measure message in block 435 and returns to block 410 to poll the next neighbor.

If the proximity measure is not less than the proximity threshold 115 in block 425, the proximity monitor 110 broadcasts a proximity measure message in block 435 and returns to block 410 to poll the next neighbor.

In some embodiments, the proximity monitor 110 flags certain entries in the neighbor list 250 for directed polling. For example, If the proximity measure for a particular neighbor is less than a predetermined threshold, the neighbor may be flagged for directed polling. The directed polling threshold may differ depending on whether the proximity measure was generated by directed polling or by estimation.

In some embodiments, the proximity monitor 110 employs a first time interval for sending repeated broadcast poll messages to identify neighbors and a second time interval for sending repeated directed poll messages to measure proximity. Wireless technologies, including UWB, have limits as to how many transmission packets that can be exchanged in a certain period of time to avoid congestion and the resulting increased error rates. In the context of proximity tracking, this limit suggests that there is a limited number of neighbors in a specified area that can be measured using directed polling within a specified period of time. As the number of neighbors the system tries to measure increases, the rate at which the measurements can be completed slows down proportionately.

In some embodiments, the proximity monitor 110 increases message efficiency by changing the first time interval for broadcast poll messages to identify neighbors or the second time interval for directed poll messages to measure proximity of neighbors depending on the environment and user behavior.

In some embodiments, the first time interval is adjusted based on the number of neighbors. As the number of neighbors increases the first time interval is increased. Increasing the first time interval can be completed without reducing tracking efficacy, because an increased number of neighbors equates to an increased number of proximity measure messages exchanged between other neighbors. An increased number of proximity measure messages makes it less likely that a new neighbor is not identified by a given proximity monitor 110, since the new neighbor is likely to be identified by a different proximity monitor 110 prior to the neighbor getting close to the given proximity monitor 110.

In some embodiments, the first time interval or the second time interval are adjusted based on the motion states of the proximity monitor 110 and the neighbors. Data from the orientation sensor 239 indicates the motion state of a proximity monitor 110, such as a movement direction and speed. In some embodiments, if a given proximity monitor 110 is moving, the first time interval may be reduced as compared to the first time interval employed if the given proximity monitor 110 is stationary. The magnitude of the time interval decrease may be proportional to the movement rate. For a moving proximity monitor 110, the second time interval may be decreased for neighbors in the direction of the movement and increased for neighbors opposite the direction of the movement.

In some embodiments, a given proximity monitor 110 broadcasts a moving message indicating its moving state. In some embodiments, the moving message includes a movement rate and direction vector. Other proximity monitors 110 may adjust the second time interval they apply to the moving neighbor to increase tracking of moving neighbors. For a given proximity monitor 110, the second time interval may be decreased for neighbors moving in the direction of the given proximity monitor 110 and increased for neighbors moving away from the given proximity monitor 110. The movement vectors may be used to estimate a time in which the proximity threshold will be violated.

For a proximity monitor 110 on the vehicle 130 the first or second time intervals are reduced when the vehicle 130 is moving. In some embodiments, the proximity threshold 115 for the proximity monitor 110 on the vehicle 130 is changed as a function of vehicle velocity. In this manner, individuals 105A-105E may be warned of the approaching vehicle 130.

In some embodiments, the proximity monitors 110 implement collision detection and mitigation techniques to reduce error rates. Initially, the proximity monitors 110 send the broadcast poll messages at a non-synchronized interval. If the broadcast polling messages from proximity units 110 collide, the affected proximity units 110 retry at different frequencies or varied delays.

In some embodiments, the proximity monitors 110 may use different channels for communicating the various messages described herein. For example, the broadcast polling, directed polling, and response signals may communicated using a primary channel, and the proximity measure message may be communicated using a back channel. In one embodiment, the primary channel is a UWB channel, and the back channel is a Wi-Fi channel. In some embodiments, the poll messages are broadcasted in real-time to a server, such as the proximity logging unit 120. We don't want this to be a mandatory feature but will be practical in many cases. The connection to the server may be a “tag” sniffing UWB traffic or WiFi traffic device.

In some embodiments, the proximity monitor 110 adjusts the proximity threshold 115 based on the movement information. For example, the proximity threshold 115 may be increased for moving proximity monitors 110.

In some embodiments, the proximity monitor 110 uses a combination of an RF pulse and a time-coordinated sound pulse. Since the time delay of the RF signal is almost instantaneous vs. the sound signal time-of-flight, accurate distances can be achieved. In addition, both the RF signal and sound can be encoded and/or communicate at different spectrums.

The use of sensor fusion with the orientation sensors 239 and the broadcast of moving states increases the efficiency of the system. For example, in an application such as a production line where individuals are sitting or standing in the same place for extended periods of time, the monitoring time intervals may be increased to reduce traffic and power consumption.

In some embodiments, the proximity monitor 110 provides user feedback responsive to detecting a proximity event. For example, the proximity monitor 110 may provide the feedback using the user interface 238, such as an audible tone, a vibration, a flashing visual indicator, a message on the display, etc. In some embodiments, the user may acknowledge the proximity event by clicking one of the input buttons on the user interface 238. In some embodiments, when the proximity monitor 110 is associated with the vehicle 130, the proximity monitor 110 sends an alarm signal to a horn buzzer, or other warning device on the vehicle 130. An alarm condition associated with a vehicle 130 may result in alarm signals issued by the proximity monitor on the vehicle 130 and by the proximity monitors 110 of the nearby individuals 105A-105E. In some embodiments, the nature of the alarm signal, such as ahte tone, loudness, duration, or the like, may be different for vehicle proximity events compared to proximity events associated with only individuals 105A-105E.

Since all of the proximity monitors 110 are simultaneously detecting proximity events, there should be multiple detections of the same proximity events (i.e., the proximity monitor 110 detect each other). Entries in the proximity event log 240 may be aggregated such that the contact duration is generated for a plurality of overlapping proximity events identifying the same proximity monitors 110.

In some embodiments, the proximity monitor 110 issues an alarm using a threshold less than the proximity threshold 115 used to identify a proximity event. For example, the proximity monitor 110 identifies and logs a proximity event using a proximity threshold 115 of 10 feet, but issues an alarm if the proximity measure falls below an alarm threshold, such as six feet. The alarm may be an audible, visual, or vibration alarm telling the user that the alarm distance has been breached.

The use of triangulation or sensor fusion data from the orientation sensors 239 allows the proximity monitor 110 to generate a map that includes the relative positioning of its neighbors. In some embodiments, the proximity logging unit 120 generates a map indicating the relative positions of the proximity monitors 110 using the proximity measure messages to perform its own triangulation or by receiving transmissions from the proximity monitors 110 including their neighbor maps. This approach allows the proximity logging unit 120 to perform activity monitoring without the use of anchors in an UWB environment. Avoiding the use of anchors reduces system complexity, overhead, deployment time, and cost. In some embodiments, the proximity monitors 110 support caching and relaying of messages to other proximity monitors 110 to extend communication beyond a single device. This approach allows the proximity monitors 110 to distribute configuration settings, system time, or other data, such as movement vectors. The proximity monitors 110 support wireless download and synchronization of all data stored. This proximity logging unit 120 may be static or moved throughout the area to collect the data. The proximity logging unit 120 can also synchronize time and configuration settings wirelessly allowing reconfigured of the proximity monitors 110 while in service.

In some embodiments, the identification tag 236, such as an RFID tag, is used to identify that a user has entered or exited from a particular region of a facility. Contact tracing may only be enabled if the user is within a particular region. For example, when a user leaves a workplace with the proximity monitor 110, the monitoring functions may be disabled to conserve power. In some embodiments, the proximity threshold 115 or the time intervals may be adjusted based on the region. For example, if the region is equipped with barriers, such as dividers in a restroom or dividers between pieces of equipment, the proximity threshold 115 may be reduced to account for presence of the barriers, thereby reducing the likelihood of generating false proximity events. The presence of an individual 105A-105E in a particular region may be detected based on a proximity event detected between the individual 105A-105E and a stationary reference point 135. The detection of the proximity event between the individual 105A-105E and the stationary reference point 135 may trigger an action, such as the enabling or disabling of proximity tracking, a change to a proximity threshold, a change in a broadcast or directed polling interval, or the like.

The information in the proximity event logs 240 stored by the proximity logging unit 120 is useful for identifying the particular individuals that may have come into contact with an individual who tests positive for a disease and the duration of the contact to allow for rapid intervention. Also, the proximity event logs 240 are useful for excluding individuals that did not have contact with the infected individual so that the facility may continue to operate safely. Only those individuals as risk need be removed from the population of the facility for safety.

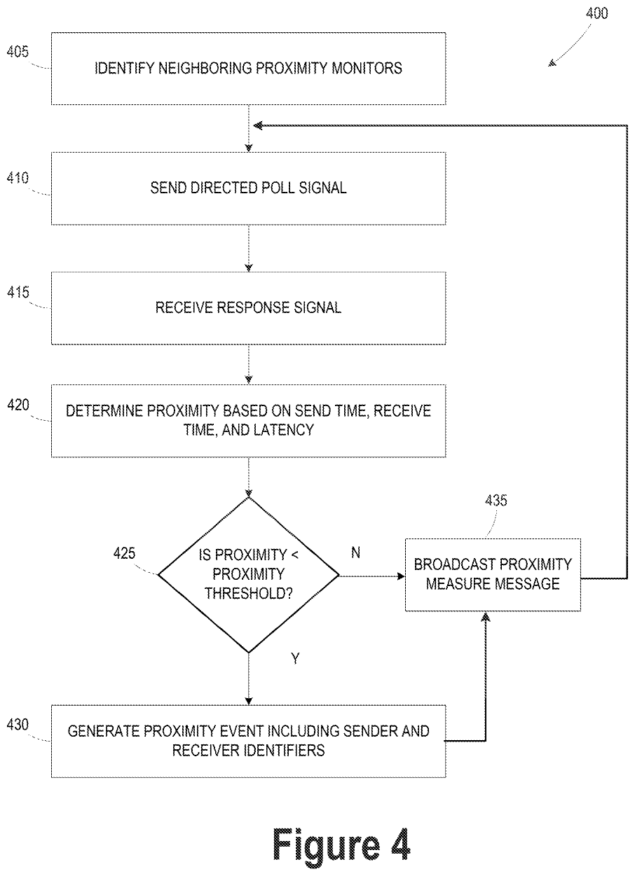

FIG. 5 is a diagram illustrating headgear 500 incorporating the proximity monitor 110. In some embodiments, the headgear is a face shield including a headband 505 and a shield 510. Placing the proximity sensor 110 on the headband 505 facilitates temperature measurement by the temperature sensor 225 on the user's head or cough detection by the cough sensor 230 due to proximity to the user's mouth. In some embodiments, the proximity monitor 110 can be integrated into other types of headgear, such as masks, hats, hardhats, or the like.

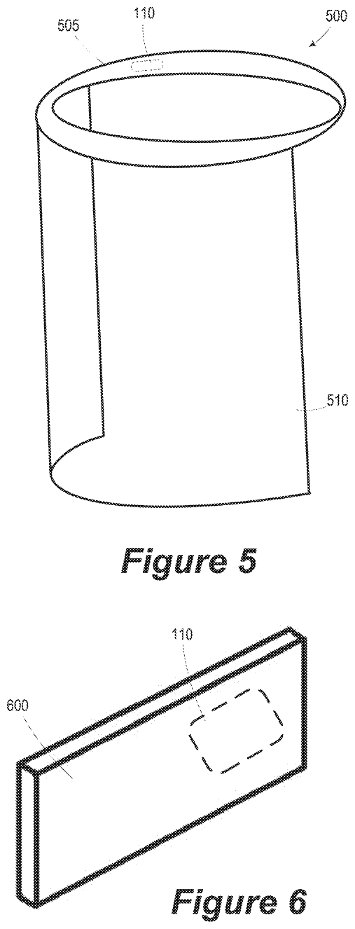

FIG. 6 is a diagram illustrating an identification (ID) badge 600 incorporating the proximity monitor 100. In some embodiments, the proximity monitor 100 is embedded into the ID badge 600 or affixed to the ID badge 600.

In the foregoing specification, specific embodiments have been described. However, one of ordinary skill in the art appreciates that various modifications and changes may be made without departing from the scope of the invention as set forth in the claims below. Accordingly, the specification and figures are to be regarded in an illustrative rather than a restrictive sense, and all such modifications are intended to be included within the scope of present teachings.

Various features and advantages of some embodiments are set forth in the following claims.

What is claimed is:

1. A proximity monitor, comprising:
a proximity detector to receive a proximity signal;
a memory; and an electronic processor to:
determine a time-of flight measurement for the proximity signal;
determine a proximity measure based on the time-of-flight;
identify a proximity event responsive to the proximity measure being less than a proximity threshold; and
store a log entry for the proximity event in the memory, the log entry identifying a first identifier associated with the proximity monitor and a second identifier associated with a sender of the proximity signal.

2. The proximity monitor of claim 1, wherein the proximity signal comprises an audible signal, and the proximity detector comprises a microphone to receive the audible signal.

3. The proximity monitor of claim 1, wherein the proximity signal comprises a radio frequency signal, and the proximity detector comprises a transceiver to receive the radio frequency signal.

4. The proximity monitor of claim 3, wherein the transceiver is to send a poll signal to a neighboring proximity monitor and receive the proximity signal from the neighboring proximity monitor, and the electronic processor is to generate the time-of-flight based on a time interval between the sending of the poll signal and the receiving of the proximity signal.

5. The proximity monitor of claim 4, wherein the electronic processor is to subtract a latency metric from the time interval to generate the time-of-flight.

6. The proximity monitor of claim 4, further comprising:
an orientation sensor to generate movement state data,
wherein the proximity monitor is to repeat the sending of the poll signal, the receiving of the proximity signal, and the generating of the time-of-flight measurement after a first time interval, and the electronic processor is to change the first time interval responsive to a change in the movement state data.

7. A method for monitoring proximity, comprising:
receiving, by a proximity detector, a proximity signal;
determining, by an electronic processor, a time-of flight measurement for the proximity signal;
determining, by the electronic processor, a proximity measure based on the time-of-flight;
identifying, by the electronic processor, a proximity event based on the proximity measure; and
storing, by the electronic processor, a log entry for the proximity event in a memory, the log entry identifying a first identifier associated with the proximity monitor and a second identifier associated with a sender of the proximity signal.

8. A proximity monitor, comprising:
a vehicle interface;
a proximity detector to receive a proximity signal;
a memory; and an electronic processor to:
determine a time-of flight measurement for the proximity signal;
determine a proximity measure based on the time-of-flight;
identify a proximity event responsive to the proximity measure being less than a proximity threshold; and
send an alarm signal over the vehicle interface responsive to identifying the proximity event.

9. A proximity monitor, comprising:
a proximity detector including a transceiver to receive a proximity signal, wherein the proximity signal comprises a radio frequency signal;
a memory; and an electronic processor to:
determine a time-of flight measurement for the proximity signal;
determine a proximity measure based on the time-of-flight;
identify a proximity event responsive to the proximity measure being less than a proximity threshold; and
modify the proximity threshold responsive to identifying the proximity event.

10. The proximity monitor of claim 9, wherein the proximity signal comprises a radio frequency signal, the proximity detector comprises a transceiver to receive the radio frequency signal, the transceiver is to send a poll signal to a neighboring proximity monitor and receive the proximity signal from the neighboring proximity monitor, and the electronic processor is to generate the time-of-flight based on a time interval between the sending of the poll signal and the receiving of the proximity signal, and to change a time interval for repeating the poll signal responsive to identifying the proximity event.
